(19) United States
(12) Patent Application Publication
(10) Pub. No.: US 2025/0073083 A1
(43) Pub. Date: Mar. 6, 2025

(54) EARMUFF WITH ADJUSTABLE NOISE REDUCTION

(71) Applicant: Casey A. Klock, Madison, WI (US)

(72) Inventor: Casey A. Klock, Madison, WI (US)

(21) Appl. No.: 18/822,130

(22) Filed: Aug. 31, 2024

Related U.S. Application Data
(60) Provisional application No. 63/663,220, filed on Jun. 24, 2024.
(60) Provisional application No. 63/535,764, filed on Aug. 31, 2023.

(51) Int. Cl. A61F 11/14; A42B 1/0188; A42B 1/06

(52) U.S. Cl. CPC A61F 11/14; A42B 1/0188; A42B 1/06

(57) ABSTRACT

An earmuff includes a first earmuff ring configured to sit against a user's head about a user's ear and a first earmuff cap including an opening and including a noise reduction member being movable relative to the earmuff ring between a first position entirely covering the opening and a second position at least partially exposing the opening, the earmuff cap having a first noise reduction rating in the first position and a second noise reduction rating in the second position that is different than the first noise reduction rating.

### CROSS-REFERENCE TO RELATED PATENT APPLICATIONS

This application claims the benefit of U.S. Provisional Application No. 63/535,764 entitled HEAD COVERING WITH HEARING PROTECTION filed on Aug. 31, 2023 and U.S. Provisional Application No. 63/663,220 entitled HEAD COVERING WITH HEARING PROTECTION filed on Jun. 24, 2024 both incorporated herein by reference in their entirety.

### BACKGROUND

Hearing protection is used to protect a person's hearing during a loud high decibel activity. Hearing protection earmuff devices that provide hearing protection are typically removed from a person's head when the loud activity has ended and replaced prior to the next high decibel activity. Certain loud activities take place in cold weather environment in which a hat is worn by the user.

### SUMMARY

In some aspects, the techniques described herein relate to an earmuff including: an earmuff ring configured to sit against a user's head about a user's ear; and an earmuff cap including an opening and including a noise reduction member being movable relative to the earmuff ring between a first position entirely covering the opening and a second position at least partially exposing the opening, the earmuff cap having a first noise reduction rating in the first position and a second noise reduction rating in the second position that is different than the first noise reduction rating.

In some aspects, the techniques described herein relate to a method for reducing ambient noise including: wearing a support having at least one earmuff ring about a user's ear; providing an earmuff cap having an opening and including a user interface; and while maintaining the earmuff ring about a user's ear moving the user interface with respect to the earmuff ring from a first position that provides a first noise reduction of the ambient noise, to a second position that provides a second noise reduction of the ambient noise, that is less than the first noise reduction.

In some aspects, the techniques described herein relate to a hat with a movable earmuff including: a head covering; and an earmuff including: an earmuff ring connected to the head covering; and an earmuff cap moveably coupled to the head covering between a first position aligned with the earmuff ring and a second position unaligned with the earmuff ring.

### BRIEF DESCRIPTION OF THE DRAWINGS

FIG. 1A is a side view of a hat with an earmuff in an aligned position.

FIG. 1B is a side view of the hat in FIG. 1A with the earmuff in an unaligned position.

FIG. 1C is an exploded view of the pivoting earmuff cap and earmuff ring of FIG. 1A and FIG. 1B.

FIG. 1D is a cross sectional view of the pivot and biasing member of the exploded pivot assembly of FIG. 1C.

FIG. 2 is an exploded view of an earmuff cap and earmuff housing member.

FIG. 2A is a cross sectional view of the earmuff cap and earmuff housing member in a locked position.

FIG. 3 is a side view of a hat with an earmuff ring.

FIG. 3A is a cross-sectional view of the hat and earmuff ring of FIG. 3.

FIG. 3B is a cross sectional view of the hat of FIG. 3 with an elastic headband.

FIG. 4 is a partially exploded isometric view of the hat and earmuff ring of FIG. 3 with an earmuff housing.

FIG. 5 is side view of a hat and the portions of the hat prior to assembly.

FIG. 6 is a force diagram illustrating the forces applied to the earmuff rings by the biasing member of the hat.

FIG. 7 includes a side view of an earmuff cap in the engaged hearing protection first position and another side view of an earmuff cap in the disengaged hearing protection second position using a button mechanism.

FIG. 8A is a an earmuff having a push activated user interface.

FIG. 8B is a view of the earmuff cap and user interface showing openings in the earmuff cap.

FIG. 8C is a cross sectional view showing the noise reduction mechanism.

FIG. 8D is a partial close up cross sectional view of a fixed cylindrical ring, rotating ring and user interface.

FIG. 8E is a top partial cross sectional view of the user interface engaging with the ring screw.

FIG. 9A is an isometric view of an earmuff with a rotating user interface in a first position.

FIG. 9B is an isometric view of the earmuff housing and user interface in a second open position.

FIG. 9C is a cross sectional view of the user interface in the second open position.

FIG. 10A is plan view of a slide mechanism for moving a user interface between first position and a second position.

FIG. 10B is a partial cross sectional view of the user interface and earmuff cap.

### DETAILED DESCRIPTION OF THE EXAMPLE EMBODIMENTS

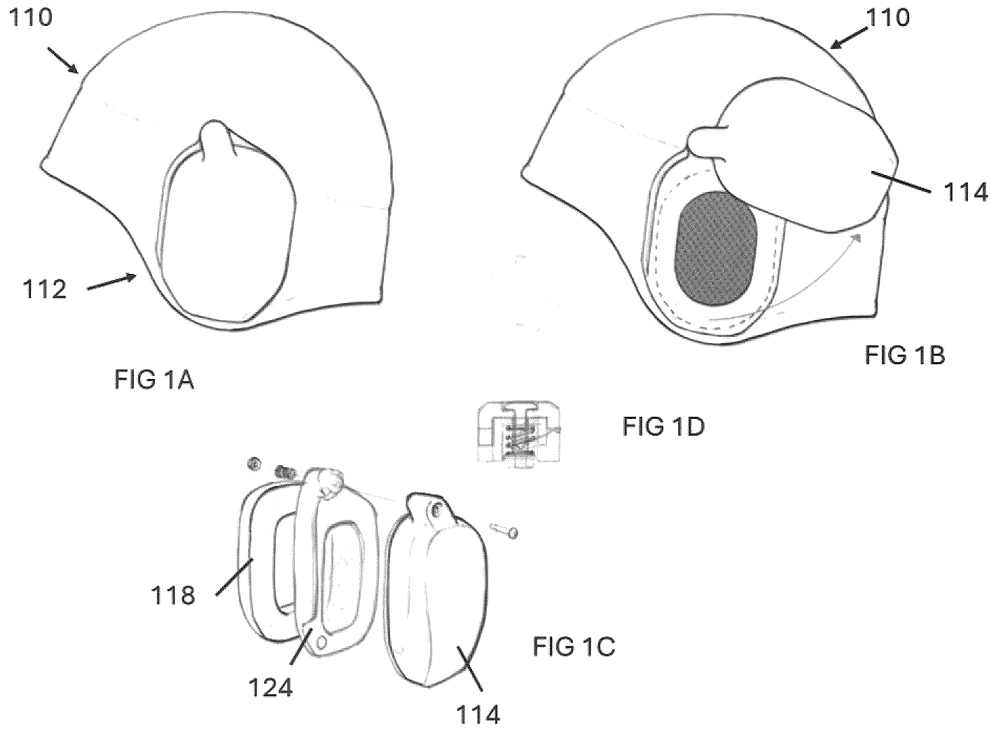

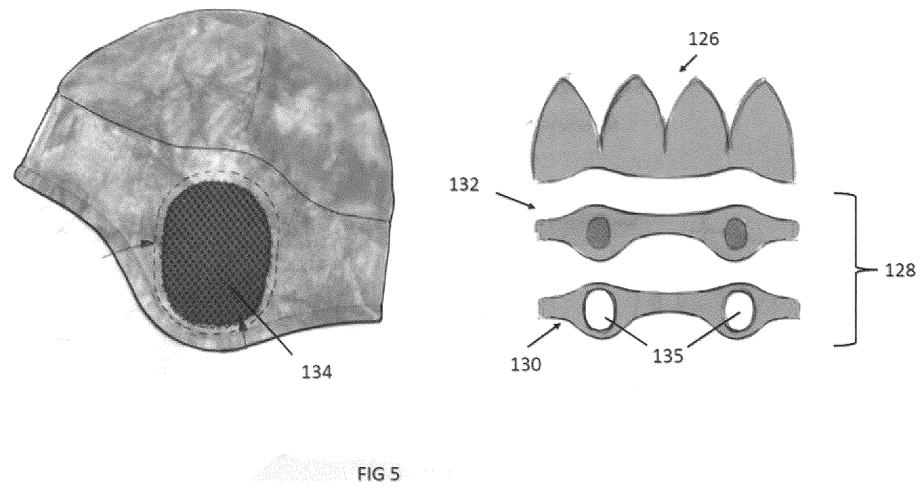

Referring to FIGS. 1A, 1B and 1C, 4 a hat 110 includes an earmuff 112 including an earmuff cap 114 movable relative to hat 110 between a first position a second position. Referring to FIG. 4 earmuff 112 includes an earmuff ring 116 positioned adjacent an interior surface 118 of hat 110. Stated another way earmuff ring 116 has a first portion adjacent the interior surface 118 and extends in a direction away from the exterior surface 120 of hat 110. Earmuff ring 116 remains in a fixed relationship to hat 110 when earmuff cap is the first position and the second position. Earmuff cap 114 remain in the same plane (XY in FIG. 1) as earmuff cap 114 is moved between the first position and the second position. In one implementation earmuff 112 includes earmuff housing base 124 and a earmuff cap 114 that is pivotally secured to earmuff housing base 124.

In one implementation earmuff cap moves in substantially the same plane relative to housing between the first and the second positions. In one implementation earmuff cap moves from a first plane in the first portion to a second plane in the second portion where the second plane is substantially parallel to and spaced the first plane. The second plane being further from the earmuff ring than the first plane as measured along an axis perpendicular to the first plane. For example, earmuff cap 114 may be immediately adjacent hat 110 in the first position and being spaced from the hat in the second position.

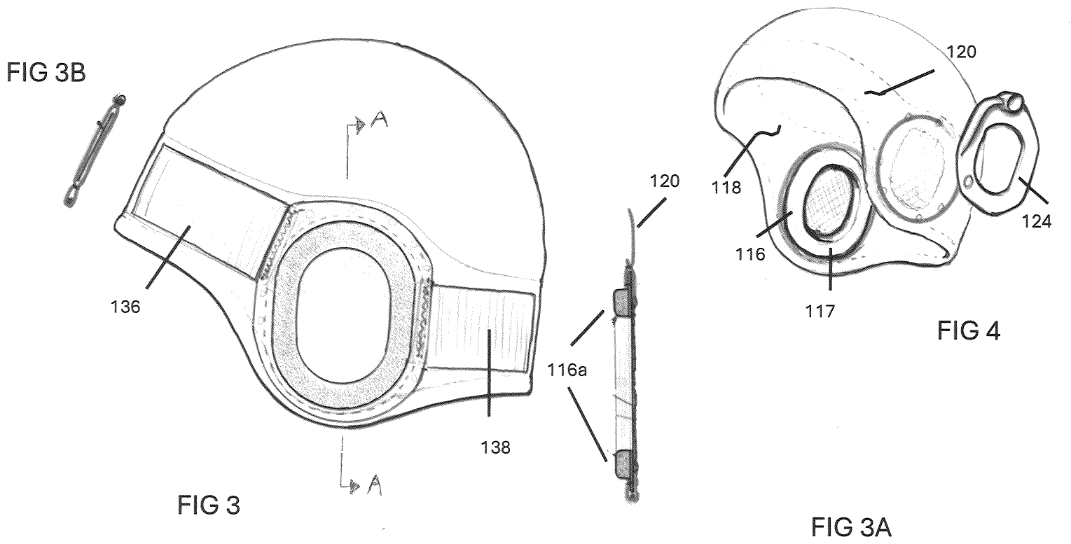

FIG. 1 and FIG. 3 show a left earmuff 112. It is understood that a second right earmuff 112b is a mirror image of left earmuff 112 when in the operational position. Right earmuff 112 includes an earmuff ring 116 and earmuff housing 124.

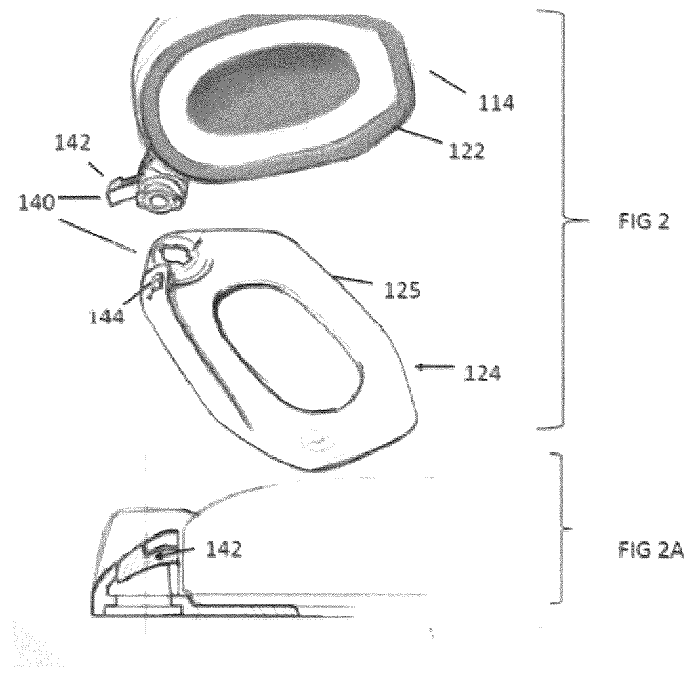

Referring to FIG. 1B and FIG. 2 earmuff 112 includes an earmuff housing 124 that is operatively secured to the outer surface of hat 110 aligned with earmuff ring 116. Earmuff cap 114 is pivotally attached to earmuff housing 124 such that in the first position an entire outer periphery of 122 of earmuff cap 114 is aligned with an entire outer periphery 117 of earmuff ring 116 and substantially about the entire outer periphery 125 of earmuff housing 124. In the second position as earmuff cap 114 is unaligned with the outer periphery 124 of earmuff housing 124.

In the first position an outer region 122 of earmuff cap 114 is aligned with earmuff ring 116. In the first position an outer region 122 of earmuff cap 114 is aligned with earmuff ring 116.

In one implementation earmuff ring 116 is secured to interior surface 118 of hat 110 with one of a mechanical fastener such as stitches or gromets, chemical adhesive, sonic welding, heat bonding or other attachment methods known in the art. Similarly, earmuff housing 124 is secured to exterior surface 120 of hat 110 with one of the methods discussed above. In one implementation earmuff housing 124 is magnetically attracted to earmuff ring 116 and maintains a position relative to hat 110 when earmuff housing 124 and earmuff ring 116 are magnetically attracted to one another.

Earmuff ring 116 is positioned within hat 110 such that when a user wears hat 110 the outer periphery of earmuff ring 116 is positioned about a user's ear such that the user's ear is completely surrounded by the outer periphery of earmuff ring 116.

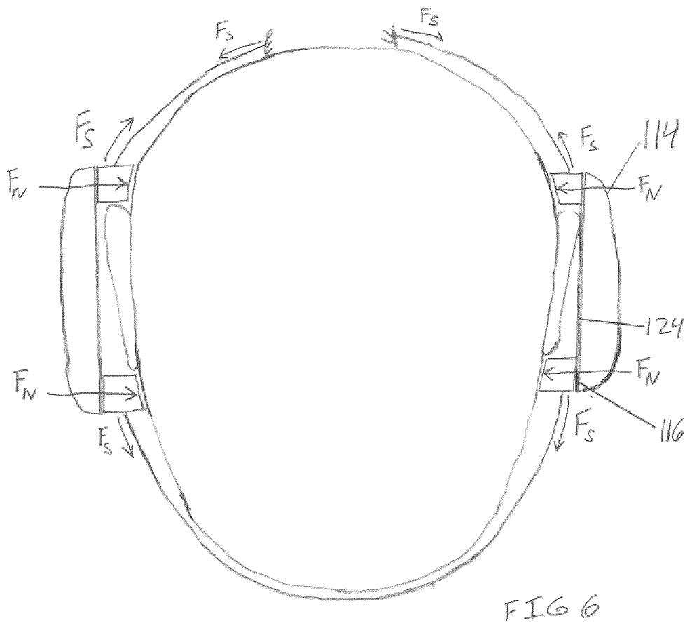

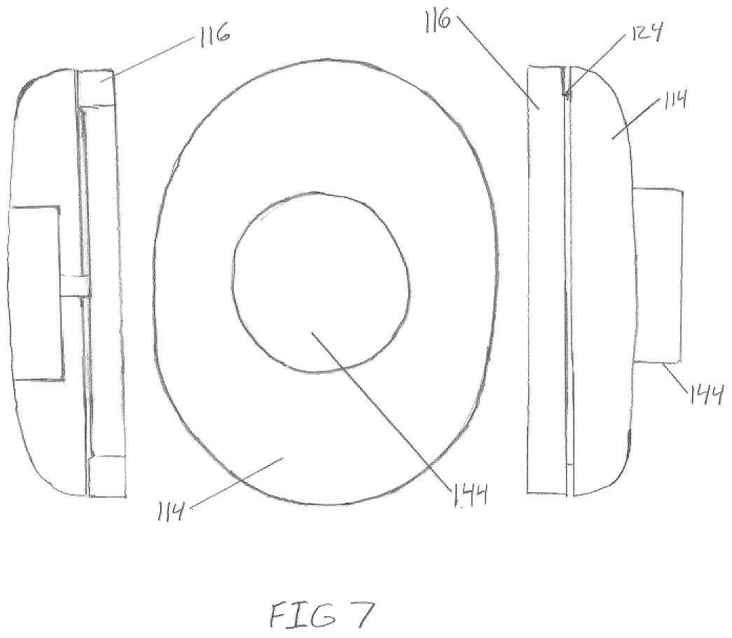

Referring to FIG. 3 and FIG. 5 hat 110 includes an upper portion 126 a hem 128 formed from an inner hem portion 130 and an outer hem portion 132. Inner hem portion 130 and outer hem portion 132 include a pair of openings 134 that are proximate earmuff ring 116 and earmuff housing 124. A mesh 134 covers the openings 135. Hat 110 includes a first member 136 extending from the earmuff ring 116 toward the front of hat 110 and a second member 138 extending from the earmuff ring toward the back of hat 110 biasing each earmuff ring 116 against a user's head proximate and about the user's ears respectively when hat 110 is worn by a user. In one implementation first member 136 is an elastic band material that is part of hat inner hem portion 130 and or outer hem portion 132. In one implementation 136 is an elastic band material that is separate from inner hem portion 130 and outer hem portion 132. Similarly, first member 136 may be integral with or separate from inner hem portion 130 and outer hem portion 132. The force applied to earmuff ring 116 against a user's head is sufficient to provide a full seal of earmuff ring 116 to the user's head. The forces applied by first member 136 and second member 138 to each earmuff ring 116 are illustrated in FIG. 6. The net force applied by first member 136 and second member 138 result in each earmuff ring 116 being pressed against a user's head to maintain a connection of earmuff 112 to a user when the earmuff 112 both when the earmuff cap 114 is in the first position and in the second position.

Referring to FIG. 3 and FIG. 6 in one implementation first member 136 and/or second member 138 is continuous single member extending about the hat from a region proximate one earmuff ring 116 to a region proximate the second earmuff ring 116. In one implementation, first member 136 and/or second member 138 is formed from at least two separate pieces each piece extending from a region proximate each respective earmuff ring toward the front/rear of the hat respectively.

Referring to FIG. 2 and FIG. 2A earmuff cap 114 is pivotally connected to earmuff ring 116 with a pivot 140 including a pivot pin extending from earmuff cap 114 that is rotated within an aperture in earmuff housing 124. A biasing member 142 fits within an opening in a portion of a raised portion of earmuff housing 124 to maintain earmuff cap 114 in contact with earmuff housing 124 as earmuff cap 114 is pivoted relative to earmuff housing 124 about the pivot pin. In one implementation a biasing member biases earmuff cap 114 against earmuff housing 124 in the first position. In one implementation a biasing member biases earmuff cap 114 to a fixed location relative to earmuff housing 124 in the second position. In this manner earmuff cap 114 remains in the second position until a user applies a force overcoming the biasing force to return earmuff cap 114 to the first position.

Other mechanisms that move the earmuff cap 114 toward and away from earmuff ring 116 are contemplated such as a sliding mechanism in which earmuff cap 114 slides along earmuff housing 124 from a first position aligned with earmuff ring 116 to a second position not aligned with earmuff ring 116. Earmuff cap 114 may slide along a linear path or a non-linear path.

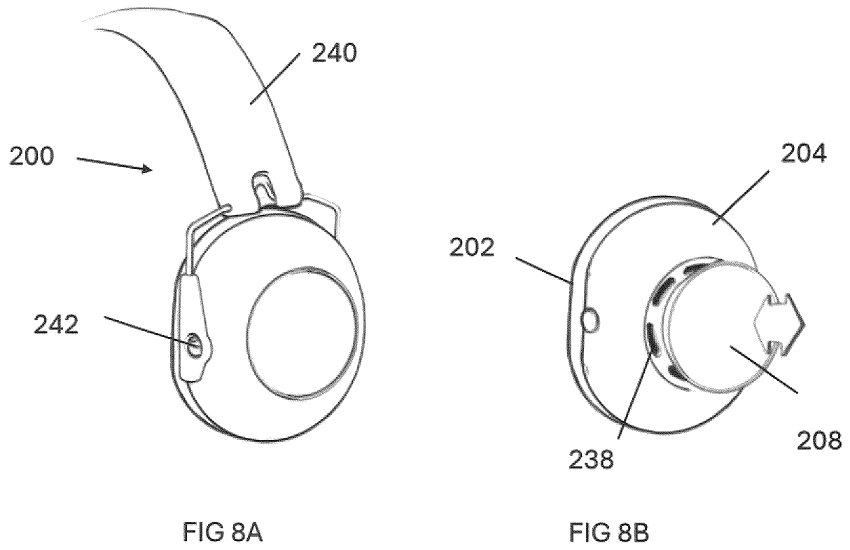

Referring to FIG. 7 a push button mechanism is used to lock and unlock earmuff cap 114 against earmuff housing 124. A user pushes the push button in a direction toward a user's ear to move the earmuff cap 114 against earmuff housing 124 in a first position in which hearing protection is maximized. (See the left side section view of FIG. 7) The user pushes the push button in the same direction to release earmuff cap 114 from contact with earmuff housing 124 to let sound enter the user's ear through the hat. (See the right side section view of FIG. 7).

In one implementation an insulated hat and/or hat with sun visor has integrated hearing protection. The insulated hat may have an earmuff ring, a plurality of earmuff ring magnets, an earmuff ring windproof/weatherproof/insulative material, an earmuff cap, a plurality of earmuff cap magnets, an earmuff cap sound reducing material, a earmuff cap weatherproof material, a strap to connect the earmuff ring and the earmuff cap, a way to connect the earmuff cap and earmuff ring assembly to the insulated hat and/or hat with sun visor for easy cleaning of the hat.

A user puts on the insulated hat and/or hat with sun visor so that the earmuff ring sits comfortably around both of the user's ears. A user ensures the earmuff caps are in the deactivated position. When the user wants additional hearing protection, the user gently pulls one or both of the earmuff caps so that the earmuff caps may then fall into the correct seated position with the earmuff cap and ring assembly to provide a NRR (noise reduction rating) of 22 dB or greater.

In one implementation a singular piece of apparel has both hearing protection and insulative properties. A hat with insulated properties and or a sun visor meant to be worn on the head that also includes an integrated means of hearing protection. Hearing protection in this description is defined as an NRR rating of 22 dB or greater when the hearing protection is deployed. In one implementation the singular piece of apparel has both hearing protection and a sun visor. In one implementation the singular piece of apparel or has insulative properties.

As described herein the hearing protection noted has a way of being reduced or removed when not needed. When the hat is being worn, but hearing protection is not desired the earmuff caps are flipped off of the earmuff rings so that hearing is maximized. The earmuff caps may be held in place when flipped up, by including but not limited to magnets.

The hat and earmuff described herein allow the hearing protection to be deployed and removed quickly when desired. In one implementation the hearing protection is able to be deployed quickly and quietly, when desired. The earmuff caps may be held in place individually and thus have to be disengaged separately by pulling the earmuff cap far enough away from the magnet(s) in the hat that it can fall into place and connect with the earmuff rings by including but not limited to magnets. The earmuff caps may be held in place together where if one earmuff cap is disengaged, the other earmuff cap automatically disengages.

The earmuff cap 114 seats itself against earmuff housing 124 when deployed. In one implementation earmuff cap 114 is secured to earmuff housing 124 by one or more magnets. In one implementation a user's ears are kept warm when the hearing protection is not deployed. When earmuff cap 114 is in a deployed or first position a user's ears are kept warm in part by the hat, earmuff ring 116 and earmuff cap 114. When earmuff cap 114 is moved to the second or not deployed position the insulated hat and earmuff ring 116 must keep the ears warm. In one implementation earmuff ring 116 has a windproof material or other material so that the ear is not directly exposed to the ambient temperature or elements.

Referring to FIG. 7 earmuff caps 114 may be moved toward and away from earmuff ring 116 in a traditional earmuff device with a standard over the head headband that biases traditional earmuff 112 against a user's ears. Traditional earmuff's that are connected to a user with a headband or the type that has a structure similar to large headphones, with a band going over the top of the head require the entire headband and earmuffs to be removed from the ears when not in use. The earmuff's provide no insulative properties to a user's ear when not deployed or in use. Referring to FIG. 7 the earmuff caps are adjacent the earmuff ring 116 to provide hearing protection in the first or employed position and then are spaced from the respective earmuff ring 116 in the second non-employed position when hearing protection is not required. Note that the band holding the earmuff ring to the user's head does not need to be removed when the earmuff cap 114 is moved from the first deployed position to the second non-deployed position.

In one implementation hat 110 is formed of a flexible material. In one implementation earmuff cap 114 is directly and moveably coupled to earmuff ring 116 between a first hearing protection position and a second non-hearing protection position.

In one implementation a hat and hearing protection includes one or more of the following feature in any and all combinations:
an insulated hat and/or hat with sun visor
an earmuff ring:
an earmuff ring magnet(s):
an earmuff ring windproof/weatherproof/insulative material:
an earmuff cap:
an earmuff cap magnet(s):
an earmuff cap sound reducing material:
an earmuff car weatherproof material:
a strap to connect the earmuff ring and the earmuff cap; and
a way to connect the earmuff cap and earmuff ring assembly to the insulated hat and/or hat with sun visor for easy cleaning of the hat.

In one implementation in use a user puts on the insulated hat and/or hat with sun visor so that the earmuff ring sits comfortably around both of the user's ears. A user ensures the earmuff caps are in the deactivated position. When a user wants additional hearing protection, the user gently pulls one or both of the earmuff caps from a disengaged to engaged position. The earmuff caps then fall into the correct seated position with the earmuff providing a NRR of 22 dB or greater.

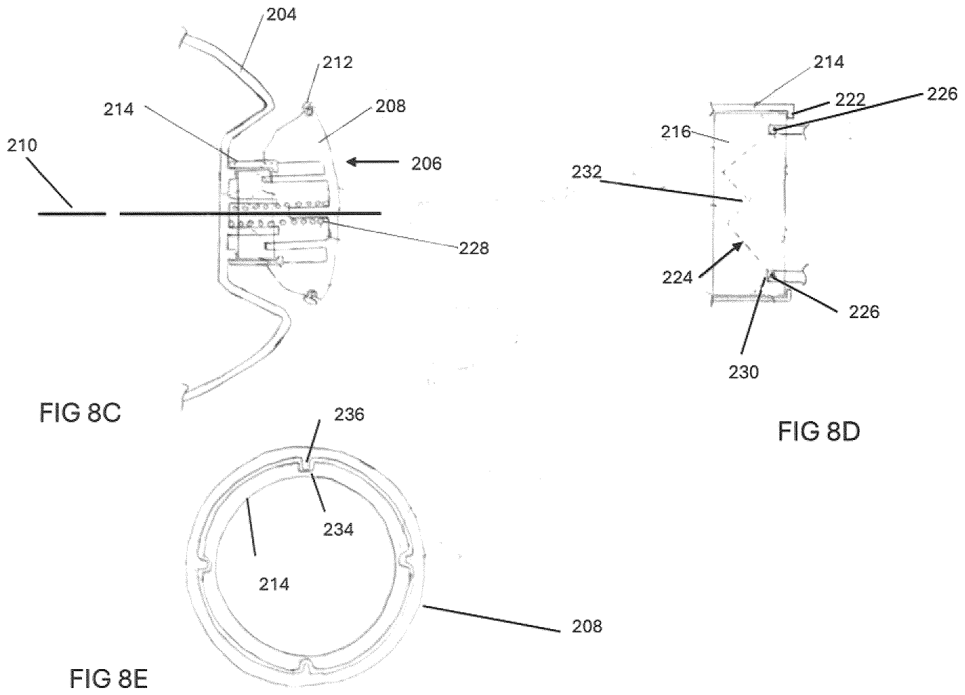

Referring to FIG. 8A an earmuff cap includes a pop-it mechanism. When the user wants hearing protection, the user simply pushes the middle portion until it clicks and locks in the closed position. When the user wants to hear better, the user simply pushes the middle portion until it clicks and is an open position. The mechanism concept is similar to those found in many pens, pop-up bathtub drain mechanism, and OXO container lids, and gas door covers on a car. There is also a hearing protection seal that ensures maximum hearing protection when in the closed position. The hearing protection seal can be of a high-viz color so that when in the open position, the seal is easy to see and indicates that the hearing protection is not engaged.

Referring to FIG. 8A-8E an earmuff 200 includes an earmuff ring 202, an earmuff housing 204 (also referred to herein as “earmuff cap”) and a noise reduction mechanism 206. Although only a single earmuff ring 202, earmuff housing 204 and noise reduction mechanism 206 is illustrated earmuff 200 includes a pair of earmuff housings that sit against each of the user's right and left ear respectively. In one implementation a headband 240 is pivotally secured to earmuff housing 204 by a pivot attachment 242 to allow a user to comfortably position each earmuff ring 202 against the user's head and about the respective user's ears. Each implantation described herein can include a first and second earmuff housing 204 to cover both the right and left ear of a user.

In one implementation noise reduction mechanism 206 includes a user interface 208 that a user pushes towards a plane defined by earmuff ring 202 along an axis 210 generally perpendicular toward the plane defined by the earmuff ring 202. User interface 208 is also referred to as a push button as the user pushes the button to move the noise reduction mechanism 206 between a first position and a second position.

A seal 212 is positioned about an outer periphery of user interface 208 to form a seal between user interface 208 and earmuff housing 204 in the first position. Referring to FIG. 8C in the second position, seal 212 does not contact both the earmuff housing 204 or user interface 208 thereby allowing ambient noise to be heard by a user. In one implementation seal 212 is secured to user interface 208.

Noise reduction mechanism 206 includes a cylindrical ring 214 (also referred to herein as a ring screw, when the cylindrical ring is screwed into earmuff housing 204) that is secured to the earmuff housing 204 so that cylindrical ring 214 is in a fixed relationship to earmuff housing 204. A moveable cam ring 216 rotates within cylindrical ring 214. Cylindrical ring 214 includes a wall 220 with an inwardly extending flange 222 to retain moveable cam ring 216 between earmuff housing 204 and flange 222. Moveable ring 216 has a groove 224 that receives a tab 226 secured to user interface 208. Moveable cam ring 216 rotates as user interface 208 is pushed along axis 210 towards earmuff ring 202. Tab 226 forces moveable cam ring 216 to rotate as tab 226 is pushed toward the earmuff ring 202. In this way biasing member 224 and tab 226 acts as a cam and follower with the tab moving linearly and movable cam ring 216 being rotated. A biasing member 228 such as a spring biases user interface 208 in a direction away from earmuff ring 202. Note that FIG. 8D shows groove 224 as a dashed hidden line, however in actuality groove 224 includes two spaced apart lines defining a groove. In implementation groove 224 includes a repeating pattern about the periphery of moveable cam ring 216.

Referring to FIG. 8E cylindrical ring 214 includes a plurality of longitudinally extending grooves 234 parallel to axis 210 having a longitudinal axis parallel to axis 210. User interface 208 includes matching ribs 236 that are slidably received within grooves 234 to prevent user interface 208 from rotating as user interface 208 is moved along axis 210. In this manner rotational movement is limited to moveable cam ring 216.

Referring to FIG. 8C in one implementation user interface noise reduction mechanism 206 includes more than one tab 226 spaced about the periphery of user interface 208 that are received within groove 224. Note that each tab 226 of the plurality of tabs 226 are positioned within a repeating pattern of groove 224. Groove 224 has defines a non-straight line path about the wall of moveable cam ring 216 including high points 230 and low points 232. Groove 224 is a channel within the of movable cam ring 216 but does not extend completely through the wall of movable cam ring 216. Each time a user pushes user interface 208 along axis 210 moveable cam ring 216 is rotated an amount such that tabs 226 are moved from high points 230 and low points 232 thereby moving noise reduction mechanism 206 between the second position and the first position respectively. Stated another way when tabs 226 are positioned at low points 232 of groove 224 the earmuff is in the first position reducing ambient noise. When tabs 226 are positioned at high points high points 230 of groove 224 the earmuff is in the second position allowing ambient noise to be heard by the user in a substantially non reduced manner.

Referring to FIG. 8B earmuff housing 204 includes an opening 238 allowing ambient noise to enter earmuff housing 204 and be heard by a user wearing earmuff 200 through earmuff ring 202. When user interface 208 is in a second position opening 238 is not sealed to the outside environment. When user interface 208 is moved to the first position, user interface 208 provides a seal between the user and the outside environment and the noise heard by the user is reduced.

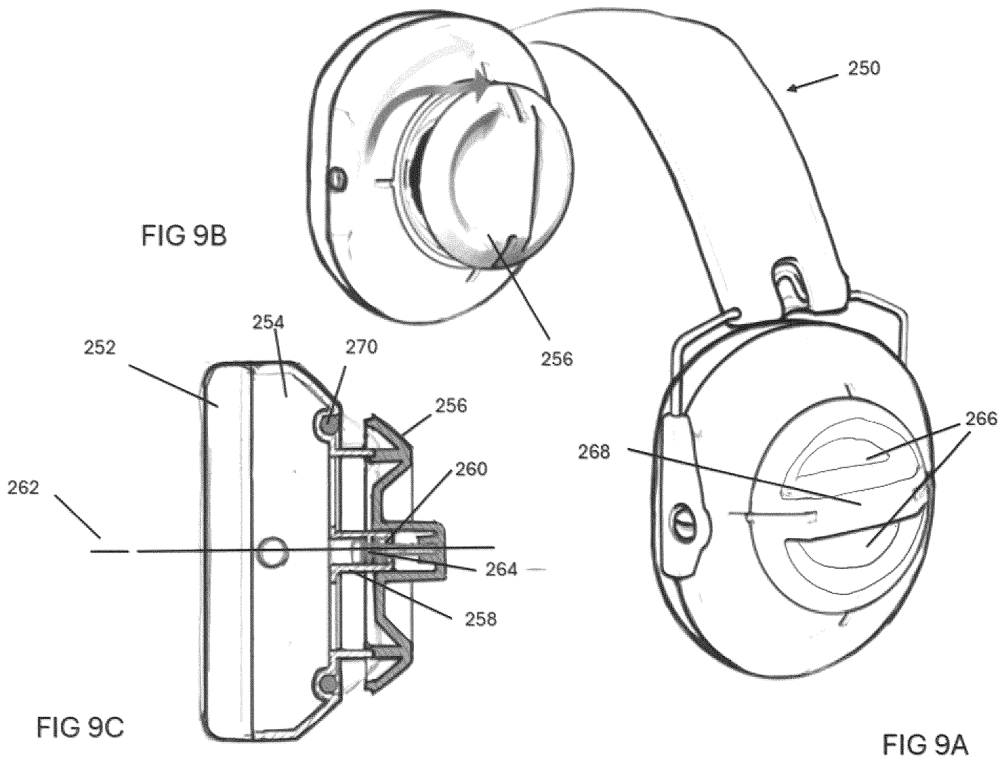

Referring to FIGS. 9A-9C an earmuff 250 includes an earmuff ring 252, an earmuff cap 254, a user interface 256. User interface 256 is rotatably secured to and a rotating mechanism 256. When the user wants hearing protection, the user simply rotates the middle portion so that the user interface is in the horizontal position and in the closed position. When the user wants to hear better, the user simply rotates the middle portion so that the user interface is in the vertical position and in the open position. The mechanism concept is similar to a screw but may not have a consistent angle so that it opens faster towards the vertical position to maximize space and hearing. There is also a hearing protection seal that ensures maximum hearing protection when in the closed position. The hearing protection seal can be of a high-viz color so that when in the open position, the seal is easy to see and indicates that the hearing protection is not engaged.

Referring to FIG. 9C earmuff cap 254 includes a stand 258 extending away from earmuff ring 252 that receives a nut 260 secured therein or a threaded region to allow user interface 256 to be threadedly moved along an axis 262 substantially perpendicular to a plane defined by earmuff ring 252. A biasing member 264 (such as a spring) biases user interface 256 in a direction away earmuff ring 252. A user rotates user interface 256 by inserting a user's fingers in depressions 266 about raised bar 268 on the exposed surface of user interface 256. A seal 270 located on one of earmuff cap 254 and user interface 256 provides a seal against outside ambient noise when user interface 256 is rotated to a closed first position. A user can hear substantially all of the ambient noise when user interface 256 is rotated to a second position where user interface 256 and earmuff cap 254 are not sealed with seal 270.

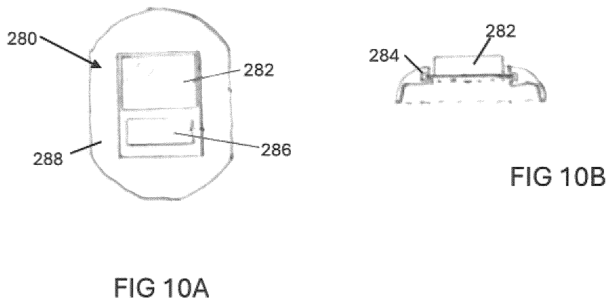

Referring to FIG. 10A and FIG. 10B in one embodiment an earmuff includes a noise reduction member 280 including a panel 282 that slides along a linear path that is not perpendicular to a plane defined by the earmuff ring. The panel 282 slides in a track 284 between a first position and a second position. Panel 282 covers an opening 286 in an earmuff cap 288 in the first position and the panel 282 does not cover at least a portion of the opening 286 in the second position.

Referring to FIGS. 1A-1C, 2 and 2A, an earmuff having a headband that is secured to housing base portion could pivot an upper portion of the earmuff cap as user interface between a first engage position and a second disengaged position. The pivot mechanisms illustrated and described herein could be used to secure the user interface to a housing base. In this implementation the earmuff ring would be secured to the housing base. A user would manually pivot the user interface about a pivot post to move the upper portion of the housing with respect to the housing base portion and earmuff ring.

In all of the embodiments provided herein the noise reduction is accomplished by mechanical means without the use of electronics such electronic noise cancelation. Rather, noise reduction is accomplished by a user manipulating a user interface to allow non reduced ambient sound to enter the earmuff housing and through the earmuff ring to allow a user to substantially hear all ambient noise. When the user interface is manipulated as described herein the openings are sealed thereby providing noise reduction of the ambient noise. In one implementation the systems described herein may include electronic noise reduction systems in conjunction with the mechanical features described herein.

In one implantation all of the user interfaces includes a sound deadening material to assist in reducing ambient noise heard by a user when the user interface is in the first engaged position.

In one implementation, an earmuff includes speaker capabilities. In one implementation when any of the mechanisms discussed herein is moved from the closed position to the open position, the sound is automatically muted, lowered in volume or paused. When the mechanism is moved from the open position to the closed position, the sound automatically plays.

Implementation 1. An earmuff comprising: an earmuff ring configured to sit against a user's head about a user's ear; and an earmuff cap including an opening and including a noise reduction member being movable relative to the earmuff ring between a first position entirely covering the opening and a second position at least partially exposing the opening, the earmuff cap having a first noise reduction rating in the first position and a second noise reduction rating in the second position that is different than the first noise reduction rating.

Implementation 2. The earmuff of implementation 1, wherein the earmuff cap includes a base portion affixed to the earmuff ring, the noise reduction member including a user interface being movable relative to the base between the first position and the second position.

Implementation 3. The earmuff of implementation 2, wherein the noise reduction member includes a user interface that moves along a linear axis between the first position and the second position.

Implementation 4. The earmuff of implementation 2, further including a seal between the base and the user interface to releasably seal the user interface to the base.

Implementation 5. The earmuff of implementation 3, wherein the user interface is a push-activated by a user moving the user interface toward the earmuff ring to both move the noise reduction member from the first position from the second position and to move the noise reduction member from the second position to the first position.

Implementation 6. The earmuff of implementation 3, including a biasing member biasing the user interface away from the earmuff ring in both the first position and the second position.

Implementation 7. The earmuff of implementation 5, including a fixed ring having a cam surfaced secured to the base, and a rotating ring rotating with respect to the fixed ring and the user interface, the rotating ring rotating from a first location on the cam surface in the first position and a second location on the cam surface in the second position.

Implementation 8. The earmuff of implementation 3, wherein the user interface does not rotate about the linear axis as the user interface is moved along the axis.

Implementation 9. The earmuff of implementation 3, wherein the user interface is rotated as the noise reduction member moves between the first and second position.

Implementation 10. The earmuff of implementation 9, wherein the user interface is threadedly attached to the earmuff cap.

Implementation 11. The earmuff of implementation 2, wherein the noise reduction member includes a panel that slides along a linear path that is not perpendicular to a plane defined by the earmuff ring.

Implementation 12. The earmuff of implementation 11, wherein the panel slides in a track, the panel covering the opening in the first position and the panel not covering at least a portion of the opening in the second position.

Implementation 13. The earmuff of implementation 2, wherein the noise reduction member includes a user interface that pivots about linear axis wherein the opening is covered in the first position and the opening is substantially exposed in the second position.

Implementation 14. The earmuff of implementation 13, further including a biasing member biasing the user interface to bias the user interface to remain in the first position and remain in the second position until a force is applied to the user interface by a user to move the user interface between the first position and the second position.

Implementation 15. The earmuff of implementation 2, wherein the earmuff cap includes an outer shell that moves relative to the earmuff ring.

Implementation 16. The earmuff of implementation 2, further including a speaker proving an audio sound that is automatically lowered in volume when the user interface is moved from the first position to the second position.

Implementation 17. A method for reducing ambient noise comprising: wearing a support having at least one earmuff ring about a user's ear; providing an earmuff cap having an opening and including a user interface; and while maintaining the earmuff ring about a user's ear moving the user interface with respect to the earmuff ring from a first position that provides a first noise reduction of the ambient noise, to a second position that provides a second noise reduction of the ambient noise, that is less than the first noise reduction.

Implementation 18. The method of implementation 17, wherein the second noise reduction of the ambient noise is substantially zero, allowing a user to hear substantially all of the ambient noise.

Implementation 19. A hat with a movable earmuff comprising: a head covering; and an earmuff including: an earmuff ring connected to the head covering; and an earmuff cap moveably coupled to the head covering between a first position aligned with the earmuff ring and a second position unaligned with the earmuff ring.

Implementation 20. The hat of implementation 19, wherein the earmuff ring remains in a first fixed position relative to the head covering when the earmuff cap is in the first position and in the second position.

Implementation 21. The hat of Implementation 19, including an earmuff housing fixed to the hat, the earmuff cap adjacent the earmuff housing in the first position and distal the earmuff housing in the second position

Implementation 22. The hat of Implementation 19, including a biasing member biasing the earmuff cap toward the earmuff ring in the first position.

Implementation 23. The hat of Implementation 19, wherein the biasing member biases the earmuff cap away from the earmuff ring in the second position.

Implementation 24. The hat of Implementation 19, covering coupler including a first portion on the earmuff cap and a second portion on the earmuff ring directly connecting the earmuff cap to.

Implementation 25. The hat of Implementation 19, wherein the head covering is formed from a flexible material.

Implementation 26. The hat of Implementation 19, wherein the head overing extends over a user's ears.

Although the present disclosure has been described with reference to example embodiments, workers skilled in the art will recognize that changes may be made in form and detail without departing from the spirit and scope of the defined subject matter. For example, although different example embodiments may have been described as including one or more features providing one or more benefits, it is contemplated that the described features may be interchanged with one another or alternatively be combined with one another in the described example embodiments or in other alternative embodiments. Not all changes in the technology are foreseeable. The present disclosure described is manifestly intended to be as broad as possible. For example, unless specifically otherwise noted, the definitions reciting a single particular element also encompass a plurality of such particular elements The following definitions provide a non-limiting implementations of the hat and hearing protection described herein.

What is claimed is:

1. An earmuff comprising:
an earmuff ring configured to sit against a user's head about a user's ear; and
an earmuff cap including an opening and including a noise reduction member being movable relative to the earmuff ring between a first position entirely covering the opening and a second position at least partially exposing the opening, the earmuff cap having a first noise reduction rating in the first position and a second noise reduction rating in the second position that is different than the first noise reduction rating.

2. The earmuff of claim 1, wherein the earmuff cap includes a base portion affixed to the earmuff ring, the noise reduction member including a user interface being movable relative to the base between the first position and the second position.

3. The earmuff of claim 2, wherein the noise reduction member includes a user interface that moves along a linear axis between the first position and the second position.

4. The earmuff of claim 2, further including a seal between the base and the user interface to releasably seal the user interface to the base.

5. The earmuff of claim 3, wherein the user interface is a push-activated by a user moving the user interface toward the earmuff ring to both move the noise reduction member from the first position from the second position and to move the noise reduction member from the second position to the first position.

6. The earmuff of claim 3, including a biasing member biasing the user interface away from the earmuff ring in both the first position and the second position.

7. The earmuff of claim 5, including a fixed ring having a cam surfaced secured to the base, and a rotating ring rotating with respect to the fixed ring and the user interface, the rotating ring rotating from a first location on the cam surface in the first position and a second location on the cam surface in the second position.

8. The earmuff of claim 3, wherein the user interface does not rotate about the linear axis as the user interface is moved along the axis.

9. The earmuff of claim 3, wherein the user interface is rotated as the noise reduction member moves between the first and second position.

10. The earmuff of claim 9, wherein the user interface is threadedly attached to the earmuff cap.

11. The earmuff of claim 2, wherein the noise reduction member includes a panel that slides along a linear path that is not perpendicular to a plane defined by the earmuff ring.

12. The earmuff of claim 11, wherein the panel slides in a track, the panel covering the opening in the first position and the panel not covering at least a portion of the opening in the second position.

13. The earmuff of claim 2, wherein the noise reduction member includes a user interface that pivots about linear axis wherein the opening is covered in the first position and the opening is substantially exposed in the second position.

14. The earmuff of claim 13, further including a biasing member biasing the user interface to bias the user interface to remain in the first position and remain in the second position until a force is applied to the user interface by a user to move the user interface between the first position and the second position.

15. The earmuff of claim 2, wherein the earmuff cap includes an outer shell that moves relative to the earmuff ring.

16. The earmuff of claim 2, further including a speaker proving an audio sound that is automatically lowered in volume when the user interface is moved from the first position to the second position.

17. A method for reducing ambient noise comprising:
wearing a support having at least one earmuff ring about a user's ear;
providing an earmuff cap having an opening and including a user interface; and
while maintaining the earmuff ring about a user's ear moving the user interface with respect to the earmuff ring from a first position that provides a first noise reduction of the ambient noise, to a second position that provides a second noise reduction of the ambient noise, that is less than the first noise reduction.

18. The method of claim 17, wherein the second noise reduction of the ambient noise is substantially zero, allowing a user to hear substantially all of the ambient noise.

19. A hat with a movable earmuff comprising:
a head covering; and
an earmuff including:
an earmuff ring connected to the head covering; and
an earmuff cap moveably coupled to the head covering between a first position aligned with the earmuff ring and a second position unaligned with the earmuff ring.

20. The hat of claim 19, wherein the earmuff ring remains in a first fixed position relative to the head covering when the earmuff cap is in the first position and in the second position.
